(19) United States
(12) Patent Application Publication
(10) Pub. No.: US 2026/0321372 A1
(43) Pub. Date: Oct. 15, 2026

(54) SYSTEM AND METHOD FOR CREATING PERSONALIZED NFC-ENABLED MEDICAL POCKETCARD

(71) Applicants: Eric Wurtenberg, Essex Fells, NJ (US); Craig Wurtenberg, Manalapan, NJ (US); Mark Zimmerman, Morris Plains, NJ (US)

(72) Inventors: Eric Wurtenberg, Essex Fells, NJ (US); Craig Wurtenberg, Manalapan, NJ (US); Mark Zimmerman, Morris Plains, NJ (US)

(73) Assignees: Eric Wurtenberg, Essex Fells, NJ (US); Craig Wurtenberg, Manalapan, NJ (US); Mark Zimmerman, Morris Plains, NJ (US)

(21) Appl. No.: 19/335,269

(22) Filed: Sep. 22, 2025

Related U.S. Application Data
(60) Provisional application No. 63/755,538, filed on Feb. 7, 2025.

(51) Int. Cl. G16H 10/65; G06K 19/07

(52) U.S. Cl. CPC G16H 10/65; G06K 19/0723

(57) ABSTRACT

One or more embodiments of the present disclosure relate to a system, method, and product for creating personalized NFC enabled medical pocket card. The mechanism includes receiving personalized user inputs from a user. Further, the mechanism includes selecting medical illustrations from a categorized library of medical illustrations. The categorized library is dynamically presented based on the received user inputs. Furthermore, the mechanism includes integrating the selected medical illustrations and personalized user inputs into a unified design for a medical pocket card. Moreover, the mechanism includes generating a print-ready file of the medical pocket card for printing a physical medical pocket card. Additionally, the mechanism includes embedding a programmed NFC chip and an oversized antenna into the printed medical pocket card. The NFC chip is pre-programmed with a unique URL or resource identifier that provides access to online educational materials, patient resources, or multimedia content.

### CROSS-REFERENCE TO RELATED APPLICATIONS

This application claims the benefit of U.S. Provisional Patent Application No. 63/755,538, filed on Feb. 7, 2025, and titled “System and method for creating personalized NFC-enabled medical pocket card” which is incorporated herein by reference in its entirety.

### BACKGROUND

### Technical Field

The present disclosure relates to the field of personalized healthcare communication tools, and in particular, relates to a system and method for creating personalized NFC-enabled medical pocket card.

### Description of the Related Art

Healthcare professionals frequently rely on physical tools and printed materials to educate patients and provide medical resources. Traditional methods include pamphlets, brochures, and business cards that offer static, general-purpose content tailored to broad medical topics. While effective for basic communication, these materials lack the ability to deliver interactive, patient-specific, or up-to-date information. As a result, they often fail to address the personalized needs of patients or the evolving requirements of modern healthcare environments.

In parallel, the integration of technology into patient care has become increasingly prevalent. Tools like patient portals, healthcare apps, and wearable devices have enhanced the accessibility of information. However, these technologies require patients to actively seek and navigate them, which may lead to underutilization, particularly among individuals who are less tech-savvy or those who prefer simpler, more tangible tools.

Further, QR code-based systems which widely used for contactless information sharing also have notable limitations that hinder their effectiveness, particularly for certain user groups. Specifically, QR codes require precise visual alignment and manual scanning via a smartphone camera, which can be difficult for individuals with visual impairments who struggle to locate or focus on the code. Additionally, users with motor or neurological impairments, such as Parkinson's disease, multiple sclerosis, or essential tremor, may find it challenging to hold a device steady enough to scan the QR code. Furthermore, the QR codes necessitate a multi-step process, scanning, interpreting on-screen prompts, and selecting links, which can be burdensome for individuals with cognitive disabilities like dyslexia or traumatic brain injuries. Moreover, the QR codes rely heavily on the visibility of the printed image, making them vulnerable to failure in low-light environments or if damaged or obstructed. These limitations underscore the need for more reliable and user-friendly alternatives that address the diverse needs of patients and healthcare professionals alike.

Near Field Communication (NFC) technology has been widely adopted in fields such as payments, transportation, and contactless information sharing. Further, the NFC's ability to establish a quick, seamless connection between devices has made it a convenient medium for exchanging data over short distances. Despite its widespread use in consumer and industrial sectors, its application in the medical field remains underexplored, particularly in the context of providing personalized patient education or real-time access to resources.

Furthermore, personalization of patient-facing materials has traditionally been limited to static customization options, such as adding a healthcare provider's contact information or basic branding. These options do not allow for on-demand tailoring of materials to specific patient needs or conditions. Additionally, the lack of multilingual support in traditional resources often hinders effective communication with patients who speak languages other than the primary language used in a given healthcare setting.

In terms of distribution, traditional printed materials often require bulk production and manual dissemination, resulting in inefficiencies and delays. Moreover, once printed, these materials are static and cannot be updated or repurposed, which poses challenges for addressing changing medical guidelines, new treatment options, or updated institutional branding.

Despite advancements in digital healthcare, there remains a gap between physical and digital resources. Current tools are either exclusively digital, requiring active patient engagement with online platforms, or entirely physical, offering limited flexibility and scalability. Therefore, there is a need for a system and method for creating personalized NFC-enabled medical pocket card that address the above-mentioned drawbacks and enable healthcare professionals to provide patients with timely, personalized, and accessible information without the constraints of traditional methods.

### BRIEF SUMMARY

One or more embodiments are directed to a system and method for creating personalized Near Field Communication (NFC) enabled medical pocket card. The medical pocket card incorporate user-provided details, including names, credentials, and practice information, along with illustrations selected from a categorized library covering topics like anatomy, medical conditions, and procedures. The design process supports multilingual text integration, ensuring the card cater to diverse audiences while maintaining professional and visually appealing layouts. Medical illustrations and text are dynamically formatted to align with the card dimensions, creating a polished and professional appearance. Users can preview and refine their designs before finalizing them for production. Each card is embedded with a pre-programmed NFC chip, which contains a unique URL or resource identifier. The chips allow recipients to access digital resources, such as patient education tools and multimedia content, by simply tapping the card with an NFC-enabled device.

An embodiment of the present disclosure relates to a system for creating personalized Near Field Communication (NFC) enabled medical pocket card. The system includes a receiving module to receive personalized user inputs from a user. The user inputs include a name, a credential, and/or a practice name. Further, the user input adheres to predefined formatting requirements, such as proper capitalization, character limits, and text alignment. In an embodiment, the receiving module facilitates multilingual options, enabling users to input text in a preferred language.

In an embodiment, the system includes a content selection module to select medical illustrations from a categorized library of medical illustrations. The categorized library is dynamically presented based on the received user inputs. Further, the categorized library is organized by topics including anatomy, medical conditions, and/or medical procedures. Further, the categorized library is enriched with metadata to enable keyword-based searching and filtering for efficient navigation. In an embodiment, once selected, the medical illustrations are dynamically adjusted to fit within predefined dimensions and layout constraints, ensuring compatibility with the card's design.

In an embodiment, the system includes a content integration module to integrate the selected medical illustrations and personalized user inputs into a unified design for a medical pocket card. The integration is performed in a manner that ensures proper alignment and formatting, resulting in a professional and visually appealing layout.

In an embodiment, the system includes a print management module to generate a print-ready file of the medical pocket card for printing a physical medical pocket card. The print-ready file is optimized for high-resolution output by applying color calibration, bleed margin adjustments, and other print settings to ensure compatibility with printing environments. Further, the print-ready file is optimized for high-resolution output by applying color calibration, bleed margin adjustments, and other print settings. Further, the print-ready file is generated in multiple formats to ensure compatibility with different production environments, such as thermal or ink-based printing systems.

In an embodiment, the system includes an embedding module to embed a programmed NFC chip and an oversized antenna into the physical medical pocket card. The NFC chip is pre-programmed with unique identifiers. The identifiers include URL(s) and/or resource identifier that provides access to online educational materials, patient resources, or multimedia content. Further, the identifiers provide access to online educational materials, patient resources, or multimedia content.

An embodiment of the present disclosure relates to a method for creating personalized Near Field Communication (NFC) enabled medical pocket card. The method includes receiving personalized user inputs from a user. The personalized user inputs include a name, a credential, and a practice name. Further, the method includes selecting medical illustrations from a categorized library of medical illustrations. The categorized library is dynamically presented based on the received user inputs. The library is organized by topics including anatomy, medical conditions, and/or medical procedures. Furthermore, the method includes integrating the selected medical illustrations and personalized user inputs into a unified design for a medical pocket card. The integration ensures proper alignment and formatting for a professional and visually appealing layout. Moreover, the method includes generating a print-ready file of the medical pocket card for printing a physical medical pocket card. The print-ready file is optimized for high-resolution output by applying color calibration, bleed margin adjustments, and other print settings to ensure compatibility with printing environments. Additionally, the method includes embedding a programmed NFC chip and an oversized antenna into the printed physical medical pocket card. The NFC chip is pre-programmed with a unique URL or resource identifier that provides access to online educational materials, patient resources, or multimedia content.

The features and advantages of the subject matter here will become more apparent in light of the following detailed description of selected embodiments, as illustrated in the accompanying FIGURES. As will be realized, the subject matter disclosed is capable of modifications in various respects, all without departing from the scope of the subject matter. This summary is not intended to identify key or essential features of the claimed subject matter, nor is it intended to limit the scope of the claimed subject matter. Accordingly, the drawings and the description are to be regarded as illustrative in nature.

### BRIEF DESCRIPTION OF THE DRAWINGS

The present subject matter will now be described in detail with reference to the drawings, which are provided as illustrative examples of the subject matter so as to enable those skilled in the art to practice the subject matter. Notably, the FIGURES and examples are not meant to limit the scope of the present subject matter to a single embodiment, but other embodiments are possible by way of interchange of some or all of the described or illustrated elements and the features depicted in the accompanying FIGURES are not to scale, and components within the figures may be shown not to scale with each other, and further, wherein:

FIG. 1 illustrates an environment having a system for creating personalized Near Field Communication (NFC) enabled medical pocket card, in accordance with an embodiment of the present disclosure;

FIG. 2 illustrates an exemplary block diagram of the system for creating personalized NFC enabled medical pocket card, in accordance with an embodiment of the present disclosure;

FIG. 3A illustrates an exemplary operational framework for creating personalized NFC enabled medical pocket card, in accordance with an embodiment of the present disclosure;

FIG. 3B illustrates an exemplary process for creating and hand-delivering the personalized pocket card onsite, in accordance with an embodiment of the present disclosure;

FIG. 3C illustrates an exemplary process for creating and sending the personalized pocket card via mail, in accordance with an embodiment of the present disclosure.

FIGS. 4A-4F illustrates various exemplary user interfaces of an application for creating personalized NFC enabled medical pocket card, in accordance with an embodiment of the present disclosure.

FIG. 5 is a flowchart illustrating a method for creating personalized NFC enabled medical pocket card, in accordance with an embodiment of the present disclosure;

FIG. 6 is an exemplary computer unit in which or with which embodiments of the present disclosure may be utilized.

Other features of embodiments of the present disclosure will be apparent from accompanying drawings and detailed description that follows.

### DETAILED DESCRIPTION

In the following description, for purposes of explanation, numerous specific details are set forth in order to provide a thorough understanding of the present disclosure. It will be apparent, however, to one skilled in the art that the present disclosure is not limited to these specific details. In other instances, structures and devices are shown in block diagram form only in order to avoid obscuring the present technology.

The terms “connected” or “coupled” and related terms are used in an operational sense and are not necessarily limited to a direct connection or coupling. Thus, for example, two devices may be coupled directly, or via one or more intermediary media or devices. As another example, devices may be coupled in such a way that information can be passed there between, while not sharing any physical connection. Based on the disclosure provided herein, one of ordinary skill in the art will appreciate a variety of ways in which connection or coupling exists in accordance with the aforementioned definition.

If the specification states a component or feature “may”, “can”, “could”, or “might” be included or have a characteristic, that particular component or feature is not required to be included or have the characteristic.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Embodiments of the present disclosure relate to a system and method for creating personalized NFC-enabled medical pocket card, designed for healthcare professionals. The card integrates user inputs such as names, credentials, and practice details with selected medical illustrations from a categorized library organized by topics like anatomy, medical conditions, and procedures. The illustrations are dynamically adjusted to fit the card's dimensions, ensuring a professional and visually appealing design. Multilingual support enables users to input text in their preferred language, with seamless integration of translations into the card layout.

High-quality card production is ensured through the validation of user inputs for accuracy, completeness, and formatting conformity. Text and illustrations are formatted using predefined rules for alignment, scaling, and spacing to maintain consistency. A live preview feature allows users to review and refine their design before finalization. The finalized design is optimized for high-resolution printing with adjustments such as color calibration and bleed margins to ensure a polished output. Additionally, the card are embedded with pre-programmed NFC chips containing unique URLs or resource identifiers. These chips enable recipients to access online resources like patient education materials, multimedia content, and professional tools by interacting with NFC-enabled devices.

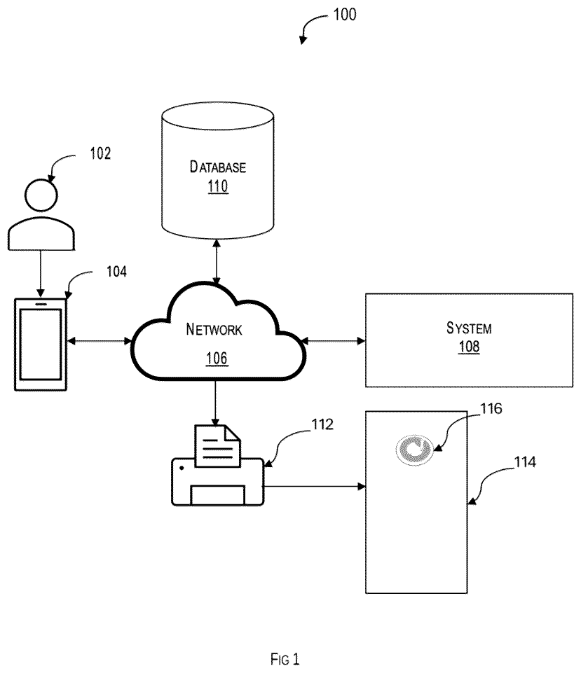

FIG. 1 illustrates an environment 100 having a system 108 for creating personalized Near Field Communication (NFC) enabled medical pocket card 114, in accordance with an embodiment of the present disclosure. In an embodiment, the environment 100 may include a user 102, a user device 104, a communication network 106, a system 108, a database 110, a printer 112, and a medical pocket card 114. The user 102 may be a healthcare professional, such as a physician, nurse, or specialist, who seeks to create personalized medical pocket card 114. The medical pocket card 114 may enhance patient education or facilitate seamless communication with patients bridging physical and digital resources. The user may interact with the system 108 to design and produce the medical pocket card 114 efficiently, ensuring they align with professional needs and preferences.

In an embodiment, the user device 104 may be any electronic device capable of communication, connecting to internet, a local network, or other devices. Further, the user device may include, but is not limited to, any electronic device capable of communication, such as AR/VR devices, smartphones, tablets, computers, kiosks, wearable devices (e.g., smartwatches, smart glasses), gaming consoles, or other electronic gadgets. The user device 104 may provide a user-friendly interface through which the user 102 may interact with the system 108. The interface may enable the user 102 to submit inputs, select options, and review previews of the card before finalizing the design. Additionally, the user device 104 may support features such as data validation or NFC testing, depending on the requirements of the system 108.

In an embodiment, the user device 104 may facilitate the user 102 to connect to the system 108 using the communication network 106. The communication network 106 may include the internet, wireless communication channels, or a local area network (LAN), facilitating the secure and seamless transmission of user inputs, design selections, and system outputs between the user device 104 and the system 108. Further, the communication network 106 may act as an intermediary that enables seamless communication and data transfer between the user device 104 and the system 108. Further, the communication network 106 may support the secure transmission of information, ensuring that interactions between the user 102 and the system 108 are both reliable and efficient. Furthermore, the communication network 106 may facilitate the system 108 to communicate with the database 110 and the printer 112. Moreover, the communication network 106 may facilitate the system 108 to communicate with external servers, sources, and/or other devices such as, but not limited to, computers, tablets, smartphones, and kiosks to enable efficient operation.

In an embodiment, the database 110 may include a storage unit. The database 110 may store various types of data, including user-provided inputs, selected content, customization preferences, and production logs. Further, the database 110 may house pre-defined resources such as medical illustrations or card templates that can be accessed by the system 108. Additionally, the database 110 may support scalability by allowing the storage of large volumes of data for future reference or reordering.

In an embodiment, the printer 112 may be utilized to produce the physical medical pocket card 114 based on the print-ready files generated by the system 108. The printer 112 may support various printing technologies, such as thermal or ink-based printing, to ensure high-resolution output. The printer 112 may be integrated with the system 108 to enable on-demand printing at live events or remote printing for large-scale production.

In an embodiment, a programmed NFC chip 116 may be embedded within the physical medical pocket card 114. The NFC chip 116 may be programmed with digital information, such as a unique URL or resource identifier. Further, the chip 116 may enable the medical pocket card to interact with NFC-enabled devices, allowing users to access digital resources or educational materials. Furthermore, the NFC chip 116 may include an oversized antenna to enhance signal strength and compatibility with a variety of devices.

In an embodiment, the user 102 accesses the system 108 via the user device 104, connecting through the communication network 106. Once connected, the user 102 may provide personalized details to the system 108. The system, based on the personalized detail, may guide the user 102 to the categorized library stored in the database 110, for selecting appropriate medical illustrations based on associated needs. The selected content and user inputs may then be integrated to create a unified design for the medical pocket card 114. The integration may include integrating the user 102 personalized details with the selected medical illustrations in a way that aligns with the medical card's layout and design requirements.

Once the integration is complete, the finalized card design may be prepared for printing. Further, the finalized card design may be visually appealing, and all text and images are properly aligned and scaled to fit within the dimensions of the medical pocket card 114. The finalized card design may then be transmitted to the printer 112 for production of the physical medical pocket card 114 with high-resolution output.

In an embodiment, following the printing process, the NFC chip 116 may be embedded into the physical medical pocket card 114. The NFC chip 116 may be programmed with a unique link or resource identifier that allows interaction with NFC-enabled devices. Such a feature may enable users to access specific online resources, such as patient education materials, multimedia content, or professional tools. Once the NFC chip 116 is embedded, the finalized medical pocket card 114 may be ready for use, providing a tangible and interactive resource that connects physical and digital elements of patient care. In an alternate embodiment, the creation of the customized pocket card 114 may be facilitated through the bulk loading of data files directly into the system 108, eliminating the need for direct input from the user 102. Such approach may enable the system 108 to efficiently process large volumes of card orders, automatically generating the customized card 114 based on the provided data.

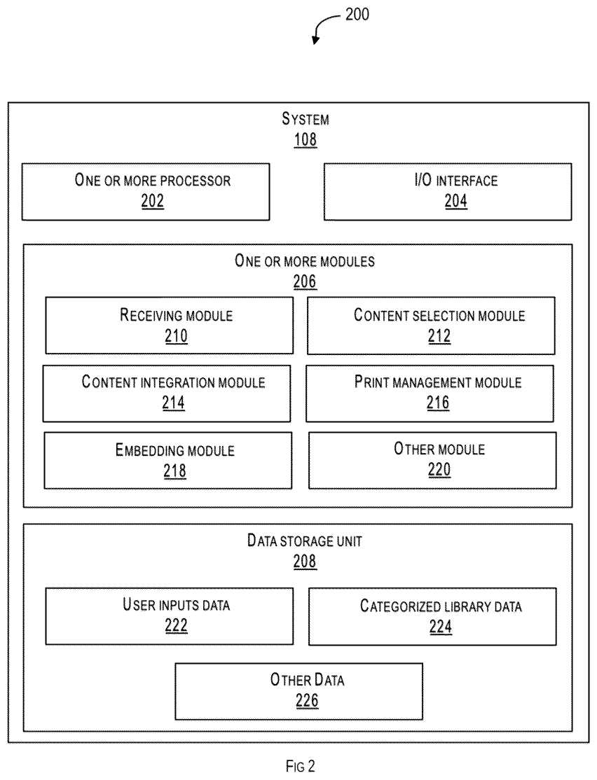

FIG. 2 illustrates an exemplary block diagram 200 of the system 108 for creating personalized NFC enabled medical pocket card 114, in accordance with an embodiment of the present disclosure. In an embodiment, the system 108 may include one or more processors 202, an Input/Output (I/O) interface 204, one or more modules 206, and a data storage unit 208. The one or more processors 202 may be implemented as one or more microprocessors microcomputers, microcomputers, digital signal processors, central processing units, state machines, logic circuitries, and/or any devices that manipulate signals based on operational instructions. Further, the I/O interface 204 may serve as the pivotal bridge connecting the internal processes of the system 108 with its external environment for facilitating the exchange of information between the system 108 and its users or external devices. Furthermore, the I/O interface 204 may contribute to the user experience by providing intuitive means for input, such as through keyboards or touchscreens, and presenting meaningful output via displays or other output devices. In an embodiment, the user interface 204 is deployed as a web application or mobile-responsive interface accessible from any web-enabled device such as a smartphone, tablet, or desktop. The input data transmitted from the user device 104 to the system 108 backend is sent over a secure communication channel, such as HTTPS with TLS encryption, to preserve confidentiality and protect against unauthorized interception or tampering during transmission

In an embodiment, the one or more modules 206 may include a receiving module 210, a content selection module 212, a content integration module 214, a print management module 216, an embedding module 218, and any other modules 220 essential or required for the working of the system 108. In an embodiment, the data storage unit 208 may include personalized user inputs data 222, categorized library data 224, and any other data 226 required for the working of the system 108. In an embodiment of the present disclosure, the one or more processors 202 and the data storage unit 208 may form a part of a chipset installed in the system 108. In another embodiment of the present disclosure, the data storage unit 208 may be implemented as a static memory or a dynamic memory. For an example, the data storage unit 208 may be internal to the system 108, such as an onside-based storage. In another example, the data storage unit 208 may be external to the system 108, such as cloud-based storage. Further, the one or more module 206 may be communicatively coupled to the data storage unit 208 and the one or more processor 202 of the system 108. The one or more processors 202 may be configured to control the operations of the one or more modules 206.

In an embodiment, the receiving module 210 may receive user inputs from the user 102 via the user device 104. The inputs may include specific details such as the user name, credentials, practice name, and/or other professional identifiers. Further, the receiving module 210 may facilitate additional user-provided preferences, such as the desired language for the content or branding elements like logos and color schemes. In an embodiment, the receiving module 210 may further include multilingual support features that allow users to select their preferred language during the input stage. The receiving module 210 may validate that the input text, including special characters or right-to-left scripts, complies with formatting rules and is properly rendered within the card layout. The selected language is incorporated into the design pipeline, ensuring that personalized user inputs are not only accepted in the selected language but also formatted and presented accurately throughout the card creation process. In an embodiment, prior to accepting user inputs, the system 108 may invoke an authentication module that verifies the identity of the user through credentials such as email verification, OTP (one-time password), or federated login. This verification step ensures that only authorized users can generate personalized medical pocket cards, and that the credentials printed on the card are traceable to a validated source.

In an embodiment, the receiving module 210 may validate the user inputs to ensure accuracy and completeness, checking for conformity with predefined formatting rules. For instance, the receiving module 210 may verify that text fields, such as the user's name or practice name, do not exceed character limits, while ensuring proper capitalization and formatting of credentials. Further, the receiving module 210 may also check for consistency across multiple fields, such as ensuring that any uploaded logos meet resolution and aspect ratio requirements. Furthermore, the receiving module 210 may prompt the user to fill in any missing mandatory fields, providing real-time feedback and error messages for incomplete or improperly formatted inputs. In an embodiment, the receiving module 210 may facilitate language-specific validation to ensure the correctness of multilingual text inputs, such as verifying that non-English characters or diacritics are supported by the system's font and card layout.

In an embodiment, the receiving module 210 may store the collected inputs in a temporary data structure for further processing, ensuring that each user session is isolated and secure. To enhance user convenience, the receiving module 210 may include features such as autosaving inputs during the customization process, facilitating the user 102 to pause and resume ongoing session without losing progress. Further, the receiving module 210 may incorporate data encryption protocols to securely handle sensitive information, such as medical titles or contact details, maintaining compliance with applicable data protection standards.

In an embodiment, the content selection module 212 may select medical illustrations or other visual content for the medical pocket card 114. Further, the content selection module 212 may provide access to a categorized library of medical illustrations stored in the database 110 that supports both metadata tagging and keyword-based search queries. The content selection module 212 may interface with the database to retrieve illustrations in real time based on user-entered keywords or filtered parameters such as anatomy type, medical specialty, or procedural category. This enables efficient access to relevant illustrations tailored to the user's context and preferences.

In an embodiment, the categorized library may be dynamically presented based on the user inputs. The user inputs, such as name, credentials, practice name, or specific keywords entered during the customization process, may be utilized to dynamically present the categorized library having illustrations that are most relevant to the user's professional identity or intended audience. For instance, if the user's practice focuses on cardiology, a categorized library having illustrations related to cardiac anatomy, conditions, or procedures may be presented. In some embodiments, the categorized library of medical illustrations may be curated for specific medical programs, events, or sponsoring organizations. For example, a healthcare sponsor may pre-approve a set of illustrations, diagrams, or condition-specific visuals that align with regulatory requirements and institutional branding. By restricting the available image pool to program-specific content, the system ensures both medical accuracy and compliance, while also allowing event organizers to deliver consistent educational messaging across all distributed cards. This curated approach distinguishes the system from general-purpose image repositories and reinforces its applicability to regulated healthcare environments.

In an embodiment, the content selection module 212 may present the categorized library in an intuitive and visually appealing interface, allowing the user 102 to browse, filter, and preview available illustrations. The categorized library of medical illustrations may include metadata tags associated with each illustration, such as topics, specialties, or patient demographics. The content selection module 212 may match these tags with the user inputs to filter and display the most relevant options. Additionally, the content selection module 212 may use filters and keyword-based search functionalities to allow users to refine their selection further. For example, a user input indicating a paediatric practice may guide the content selection module 212 to display illustrations tailored to paediatric anatomy or child-friendly designs, ensuring alignment between the visual content and the user's specific professional context.

In an embodiment, the content selection module 212 may facilitate the user 102 to preview how selected illustrations will appear on the medical pocket card 114. The preview may display the image within the card's dimensions, providing real-time feedback on placement and compatibility with other design elements, such as text or logos. To ensure proper visual quality, the content selection module 212 may validate the selected illustrations against predefined requirements, such as resolution, aspect ratio, and file compatibility. If an illustration does not meet the required standards, the content selection module 212 may alert the user 102 and provide suggestions or alternative images from the library.

In an embodiment, the content selection module 212 may provide advanced filtering options to enhance the user's experience. For example, the user 102 may filter illustrations by subcategories, medical specialties, or relevance to specific patient demographics. Metadata associated with each illustration, such as tags, descriptions, or keywords, may support more refined searches. Additionally, the content selection module 212 may facilitate a feature for the user 102 to mark certain images as “favourites” for quick access in future sessions.

In an embodiment, the content selection module 212 may also offer personalization options for the selected illustrations. The user 102 may adjust properties such as size, orientation, or placement within predefined areas of the card layout. The content selection module 212 may ensure that all adjustments comply with the card's design rules, such as maintaining margins and avoiding overlap with text fields. Furthermore, the content selection module 212 may support the selection of multiple illustrations for inclusion on different areas of the card, such as front and back, enabling comprehensive customization.

In an embodiment, the content selection module 212 may incorporate multilingual support, facilitating the user 102 to view image descriptions, category names, and search results in their preferred language. Such a feature may enhance usability for users from diverse linguistic backgrounds. Further, the content selection module 212 may ensure that any user-selected preferences, such as themes or branding elements, are taken into account when presenting the content library. In an embodiment, the content selection module 212 may also maintain a log of the user's selections, storing the data securely for future reference or modifications. The logs may facilitate the users to revisit their choices or make changes without starting the selection process anew.

In an embodiment, the content integration module 214 may integrate the personalized inputs provided by the user 102 with the selected medical illustrations to create a unified design for the medical pocket card 114. Further, the content integration module 214 may assemble all the individual elements into a cohesive layout, ensuring that the final design adheres to predefined formatting and aesthetic guidelines. Furthermore, the content integration module 214 may process inputs such as the user's name, credentials, practice name, and other professional identifiers, along with the chosen visual content to generate a visually balanced and professional design. In an embodiment, the content integration module 214 may utilize formatting rules to align and scale all components of the card design. For instance, the content integration module 214 may ensure that text inputs, such as the user's name or credentials, are displayed in appropriate fonts, sizes, and styles while maintaining consistency with the overall card layout. Further, the content integration module 214 may handle the placement of selected medical illustrations, ensuring that images fit within designated areas of the card without distortion or overlap with text. Additionally, the content integration module 214 may apply spacing rules to maintain visual clarity and ensure that all elements are properly aligned with the card's margins and boundaries.

In an embodiment, the content integration module 214 may dynamically adjust the layout to accommodate user preferences or constraints. For example, if the user 102 selects a large amount of text or multiple illustrations, the content integration module 214 may automatically resize or rearrange the elements to optimize the design while preserving readability and visual appeal. Further, the content integration module 214 may also provide real-time feedback to the user 102 by generating a live preview of the card design, facilitating the user 102 to review and make adjustments before finalizing the layout.

In an embodiment, the content integration module 214 may support multilingual customization by seamlessly incorporating translated text into the card design. Based on the user's selected language, the content integration module 214 may apply appropriate fonts, character sets, and formatting rules to ensure the text appears correctly and maintains compatibility with the card layout. For languages with special characters or right-to-left text alignment, the content integration module 214 may automatically adjust the layout to accommodate these requirements.

In an embodiment, the content integration module 214 may facilitate the incorporation of branding elements into the card design. For instance, if the user 102 provides a logo or selects specific color schemes, the content integration module 214 may integrate such elements into the layout while ensuring consistency with the overall design. Further, the content integration module 214 may apply branding guidelines, such as maintaining the logo's aspect ratio or ensuring that colors do not clash with text or images.

In an embodiment, the content integration module 214 may include error-checking capabilities to ensure the final design meets all quality standards. Further, the content integration module 214 may verify that all text is properly aligned, no elements exceed the card's dimensions, and the selected images retain their resolution and clarity. If any issues are detected, the content integration module 214 may prompt the user 102 to make corrections or suggest automatic adjustments to resolve the problems.

Accordingly, the content integration module 214 may automatically adjust the placement, scale, and alignment of both the personalized inputs and selected illustrations to fit within the predefined spatial boundaries of the card layout. This dynamic layout adjustment ensures visual clarity and prevents content clipping, regardless of variations in text length, language script, or image proportions. The system 108 may apply aspect-ratio preservation, adaptive font scaling, and margin rebalancing to optimize layout across different card templates or use cases. In an embodiment, the content integration module 214 may generate a finalized digital version of the medical pocket card 114, ready for printing or further processing. The finalized digital version may include all integrated elements, formatted to match the specifications required by the printer or subsequent modules. Further, the content integration module 214 may ensure that the finalized design captures all user preferences and selections accurately.

In an embodiment, the print management module 216 may generate a print-ready file of the medical pocket card 114 for printing a physical medical pocket card. The print-ready files may ensure that the unified design for the medical pocket card is accurately and efficiently transformed into a format suitable for physical production. Further, the print management module 216 may be the bridge between the digital design and the physical output, ensuring that all aspects of the card are optimized for high-quality printing. In an embodiment, prior to generating the final print-ready output, the system 108 may display a real-time preview of the unified digital design to the user. This preview reflects the final layout, including personalized inputs, selected illustrations, and formatting adjustments, allowing the user to review and confirm the appearance before proceeding with printing or NFC programming.

In an embodiment, the print management module 216 may first process the finalized card design to apply print-specific adjustments. The adjustments may include ensuring proper color calibration to maintain consistency between the on-screen design and the printed output. Further, the print management module 216 may account for bleed margins, which involve extending the design slightly beyond the card's edges to avoid unprinted gaps after trimming. Additionally, the print management module 216 may adjust the resolution of images and text to ensure they meet the required standards for high-resolution printing.

In an embodiment, the print management module 216 may generate the print-ready file in a format compatible with various printing systems, such as PDF, PostScript, or specialized formats for thermal and inkjet printers. Further, the print management module 216 may support generating files for single-card printing or batch production, depending on the user's needs. To ensure flexibility, the print management module 216 may facilitate the user 102 to choose specific file formats or printing configurations, such as double-sided printing or specific paper types.

In an embodiment, the print management module 216 may incorporate error-checking mechanisms to validate the print-ready file before it is sent to the printer 112. The validations may include ensuring that no text or images extend beyond the card's margins, that the NFC chip 116 embedding area is clearly marked, and that all elements are positioned correctly. If any errors are detected, the print management module 216 may alert the user 102 and provide suggestions for resolving the issues, ensuring that the final output meets the desired quality standards.

In an embodiment, the print management module 216 may also optimize the print-ready file for specific production environments. For example, the print management module 216 may adjust settings for printers that require specific paper sizes, ink density levels, or printing speeds. Additionally, the print management module 216 may provide options for selecting the number of copies, specifying unique identifiers for each card (e.g., barcodes or QR codes), or configuring additional printing requirements based on the user's preferences.

In an embodiment, the print management module 216 may support advanced printing workflows, such as print-on-demand capabilities. For live-event scenarios, the print management module 216 may streamline the process to enable rapid production of high-quality card 114 in real time. In such cases, the print management module 216 may ensure that all configurations, including NFC embedding instructions, are automatically included in the print-ready file to facilitate seamless integration with the embedding process.

In an embodiment, the print management module 216 may store a digital copy of the print-ready file for future reference or reprinting. The storing of the digital copy may allow users to retrieve previous designs and print additional copies without having to repeat the customization and design process. Furthermore, the print management module 216 may ensure the secure storage of such files, protecting sensitive user data and designs from unauthorized access.

In an embodiment, the embedding module 218 may embed a programmed NFC chip 116 into the printed physical medical pocket card. The embedding module 218 may ensure that the NFC chip 116 is securely integrated within the medical pocket card 114 while maintaining its functionality and compatibility with a wide range of NFC-enabled devices. The embedding process may be precisely controlled to ensure proper placement of the NFC chip 116, aligning with the card's design and ensuring that it does not interfere with the card's visual elements or structural integrity. In an embodiment, the embedding module 218 may handle the programming of the NFC chip 116 prior to its integration into the card. The NFC chip 116 may be (pre) programmed with a unique URL or resource identifier that links to specific online resources, such as patient education materials, multimedia content, or professional tools. The embedding module 218 may facilitate customization of the programmed content based on user preferences, ensuring that the NFC functionality aligns with the intended purpose of the card. Additionally, the embedding module 218 may initiate a locking operation on the NFC chip 116 immediately after the chip is successfully programmed, using chip-level write protection protocols or OTP (One-Time Programmable) flags, thereby preventing any subsequent rewriting or tampering. This post-programming lock ensures that the unique URL or resource identifier stored on the chip remains immutable, thereby preserving the integrity and authenticity of the linked digital content. In an embodiment, the NFC may be embedded into the physical medical pocket card 114 during the printing process. In an embodiment, the NFC may be embedded into the physical medical pocket card 114 or after the printing process.

In an embodiment, the embedding module 218 may include mechanisms for securely attaching the NFC chip 116 to the card substrate. The attaching may include techniques such as lamination, adhesive bonding, or insertion into a pre-designed slot or cavity within the card. The embedding module 218 may ensure that the NFC chip 116 is positioned in a way that optimizes its signal strength and compatibility with various smartphone models, accounting for variations in NFC reader placements. For instance, the embedding module 218 may embed the chip 116 in an oversized antenna region to enhance signal reliability and ensure seamless interactions with NFC-enabled devices. In an embodiment, the embedding module 218 may conduct quality checks to verify the functionality and durability of the NFC chip 116 after integration. Such checks may include testing the NFC chip 116 ability to establish a connection with compatible devices and ensuring that the programmed content can be accessed without errors. Further, the embedding module 218 may perform stress tests to confirm that the chip remains operational under normal usage conditions, such as bending or flexing of the card.

In an embodiment, the embedding module 218 may support batch production by enabling the integration of multiple NFC chips into a series of card 114 in an automated process. The embedding module 218 may coordinate with the printer 112 and other components of the system 108 to synchronize the embedding process with the card printing workflow. The embedding process may ensure that each card is correctly aligned with its respective NFC chip 116, maintaining consistency and accuracy during high-volume production. In an embodiment, the embedding module 218 may include an interface for monitoring and managing the embedding process. Such an interface may provide real-time updates on the status of the embedding operations, such as the number of completed card 114, error notifications, or maintenance requirements. Further, the embedding module 218 may generate detailed logs for record-keeping, ensuring traceability of each card's production and embedding details.

In an embodiment, the embedding module 218 may support advanced customization options for NFC functionality. For instance, it may allow the user 102 to configure dynamic NFC links that can redirect to updated resources over time or enable the chip to store multiple identifiers for accessing various types of content. Such capabilities may enhance the versatility of the medical pocket card 114, making them adaptable to evolving user needs and contexts.

In some embodiments, the oversized NFC antenna integrated into the medical pocket card may be dimensioned larger than conventional NFC antennas used in standard identification or payment cards. This design enables enhanced tag detection reliability in clinical environments, where users such as patients or healthcare providers may have reduced dexterity, limited device alignment accuracy, or where the card is presented through protective sleeves. By increasing the effective read range and tolerance for misalignment, the oversized NFC antenna improves first-attempt read success and thereby enhances usability and accessibility in real-world healthcare settings.

In an alternate embodiment, the print management module 216 may print directly onto a pre-programmed blank. The pre-programmed blank may already include an embedded NFC chip 116 and pre-programmed URL. The personalized card design, including text and selected illustrations, may be applied to the blank with high precision. The system 108 may ensure that the design is aligned with the embedded NFC chip 116, maintaining both visual quality and NFC functionality.

In an embodiment, the pre-programmed blanks may be fully compatible with the printing system and may ensure seamless integration of the user's customized design. Such integration may allow the medical pocket card 114 to be completed and delivered in its final form, ready for immediate use. By utilizing pre-programmed blanks, the system 108 may enhance production efficiency while maintaining the quality and functionality of the personalized NFC-enabled medical pocket card 114.

In an embodiment, any other module 220 may include additional components or functionalities that complement the primary modules in the system 108, enhancing the overall customization, production, and usability of the medical pocket card 114. These modules may support auxiliary tasks such as user support, advanced customization, analytics, or workflow optimization, ensuring a seamless and comprehensive experience for the user 102. In an embodiment, any other module 220 may include an analyzer module to track and analyze system usage, providing insights into user preferences and behaviors. The analyzer module may gather anonymized data on frequently selected medical illustrations, common customization patterns, or preferred languages. Such insights may help improve the system by tailoring content libraries, refining user interfaces, or introducing new features based on user demand. Further, the analyzer module may generate reports for users, such as summaries of completed projects, which can aid in tracking their professional branding or patient education efforts.

In an embodiment, any other module 220 may include a multilingual support module that may facilitate the system 108 to cater to diverse linguistic needs. While the core system 108 may already offer basic multilingual features, the multilingual support module may extend functionality by providing language-specific templates, support for lesser-known languages, or the ability to automatically translate user inputs into multiple languages for broader accessibility. Additionally, the multilingual support module may assist users in adapting their content to regional preferences or cultural nuances.

In some embodiments, the system may further include an analytics module configured to record and process aggregate usage data of the distributed NFC-enabled medical pocket cards. The analytics module may track interactions such as the total number of NFC taps, timestamps of access events, and general geographic or practice-level usage patterns. Importantly, the analytics module may be designed to exclude collection of personally identifiable information (PII) or protected health information (PHI), thereby maintaining compliance with applicable privacy regulations. The collected data may be aggregated and presented in the form of reports or dash-boards for healthcare sponsors, administrators, or event organizers. Such aggregate, non-PHI analytics enable evaluation of program reach, user engagement, and educational impact, thereby providing measurable insights into the effectiveness of the distributed medical pocket cards.

In some embodiments, the system may be configured to perform personalization and delivery of the medical content in less than 30 seconds from the point of user interaction or event initiation. This rapid throughput allows the system to be effectively deployed in live event settings, such as medical conferences, where on-the-fly generation and sharing of personalized cards is crucial. Such low-latency operation provides a technical improvement over conventional NFC-based card printing or generic personalization tools, which often involve significantly higher processing times.

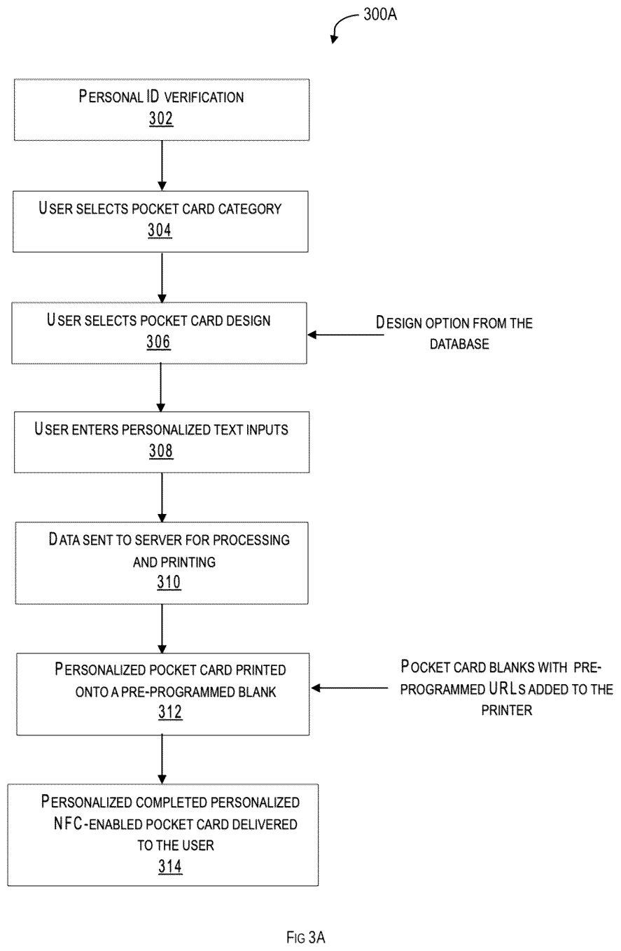

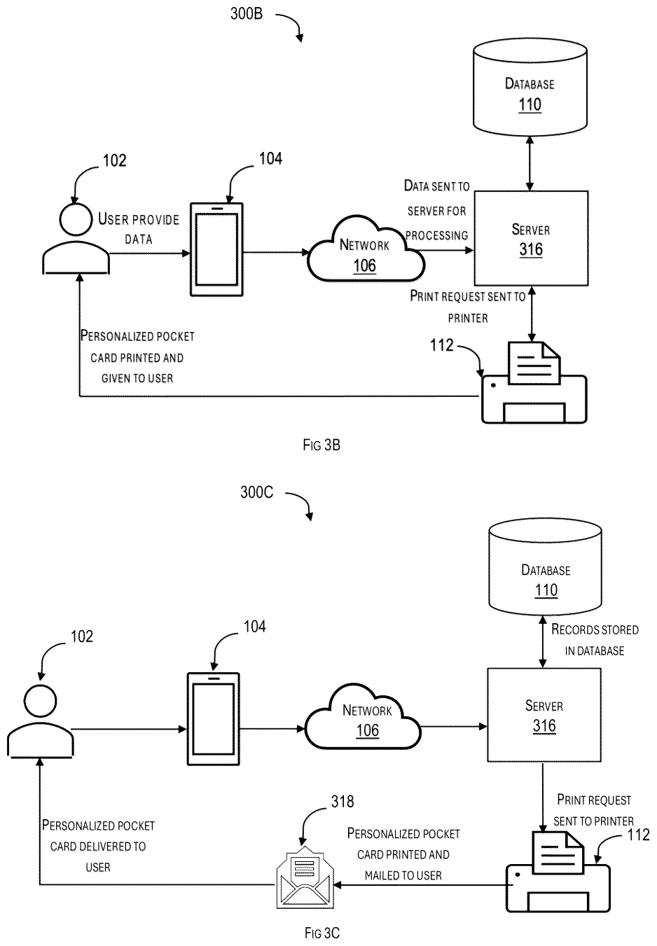

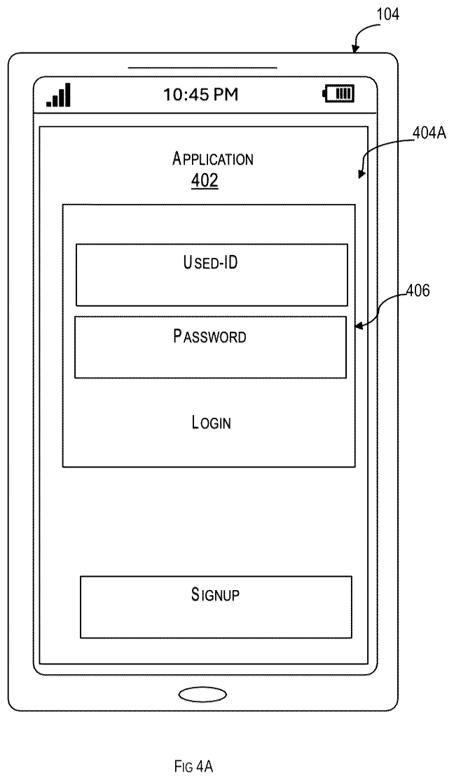

FIG. 3A illustrates an exemplary operational framework 300A for creating personalized NFC enabled medical pocket card 114, in accordance with an embodiment of the present disclosure. In an embodiment, the user 102 may enter their personal ID for verification, as shown by 302. The system 108 may authenticate the user 102 by validating the provided credentials against a secure database. The authentication step may ensure that only authorized users gain access to the card customization platform, maintaining the security and integrity of the system. Further, the user 102 may select a pocket card category, as shown by 304. The system 108 may present a variety of categories, such as professional cards for healthcare branding, patient education cards, or promotional cards for medical campaigns. Each category may include predefined templates and options tailored to the specific purpose of the card. The user 102 may browse through the categories and select one that aligns with their intended use.

Furthermore, the user 102 may select a design from the available options, stored in the database 110, within the chosen category, as shown by 306. The designs may include variations in layout, artwork, and color schemes, allowing the user to select a style that suits their preferences. Once a design is selected, the system 108 may verify it to ensure that it complies with technical requirements, such as compatibility with the card dimensions, text areas, and image placement. If any issues are detected, the system 108 may prompt the user 102 to adjust the selection or choose an alternative design.

Moreover, the user 102 may enter personalized text inputs, such as their name, credentials, practice name, or other professional details, as shown by 308. The system 108 may validate these inputs for accuracy, completeness, and adherence to formatting rules, such as proper capitalization and character limits. The personalized text may then be integrated with the selected design, ensuring that the final layout is visually appealing and professional.

Additionally, the data, including the selected design and personalized text inputs, may be sent to the server for processing and printing, as shown by 310. The server may compile the inputs into a print-ready file optimized for production. If the system 108 utilizes pre-programmed blanks, the server may also associate the design with the pre-programmed URL embedded in the blanks. This ensures that the NFC functionality is seamlessly integrated during the printing process.

In an embodiment, the personalized pocket card 114 may be printed onto a pre-programmed blank, as shown by 312. The pre-programmed blank may already include the NFC chip 116 with a pre-programmed URL or resource identifier. During the printing process, the design, including the personalized text and selected layout, may be applied to the blank in high resolution. The system 108 may also ensure that the alignment of the printed content matches the embedded NFC chip 116 location to maintain functionality. Further, the completed personalized NFC-enabled pocket card 114 may be delivered to the user 102, as shown by 314. The delivery process may include packaging the card securely and dispatching it to the user's specified address or making it available for collection at an event or designated location. Upon receipt, the user 102 may utilize the card for its intended purpose, such as providing interactive patient education or enhancing professional branding, by leveraging the embedded NFC functionality. In certain implementations, the system may be optimized for rapid throughput in live-event environments, where attendees expect immediate access to customized materials. The personalization-to-delivery cycle for producing the NFC-enabled medical pocket card may be completed in under thirty seconds from the time the user provides inputs to the time the physical card is delivered. This high-throughput capability differentiates the system from traditional personalization processes, ensuring scalability for medical conferences, health fairs, and other live events where large numbers of users may require on-demand customization within limited timeframes.

FIG. 3B illustrates an exemplary process 300B for creating and hand-delivering the personalized pocket card 114 onsite, in accordance with an embodiment of the present disclosure. FIG. 3C illustrates an exemplary process 300C for creating and sending the personalized pocket card 114 via mail, in accordance with an embodiment of the present disclosure. For the sake of brevity FIG. 3B and FIG. 3C have been explained together.

In an embodiment, in an onsite scenario, as illustrated in FIG. 3B, the user 102 may access a server 316 via the user device 104, such as a tablet, computer, or kiosk. The communication network 106 may facilitate the communication between the user device 104 and server 316. The user 102 may provide personalized inputs, including their name, credentials, and practice details, and select medical illustrations to be included on the pocket card 114. The inputs may be transmitted to the server 316 for processing. The finalized card design, optimized for high-resolution printing, may then be sent to the connected printer 112 at the same location. Once printed, the personalized pocket card 114 may be hand-delivered to the user 102 onsite, allowing for immediate access and use. Such a process may particularly be suited for live events, such as medical conferences or trade shows, where rapid customization and delivery are critical.

In an embodiment, in a mailed delivery scenario, as illustrated in FIG. 3C, the user 102 may provide inputs and selections through the user device 104, which are transmitted to the server 316 via a communication network 106. After processing, the finalized design may be sent to the printer 112, which may be located at a production facility. The completed personalized pocket card 114 may then be prepared for shipping and delivered to the user via mail 318. Such process facilitates for convenient remote access, enabling users 102 to create card 114 from any location and receive them at their preferred address. In an exemplary workflow for off-site fulfillment, a user may complete the personalization process through a web portal or mobile application, selecting medical illustrations, entering credentials, and choosing preferred language. The personalization data is then transmitted to a fulfillment center, where a pre-programmed NFC card from an inventory is matched with the user's selected design. The fulfillment system may apply the personalized print layer onto the pre-programmed card, perform quality checks to ensure NFC functionality and alignment with the printed design, and then package and ship the completed card to the designated address. This workflow enables scalable remote distribution of customized NFC-enabled medical pocket cards without requiring the user to be physically present at an event site.

In an embodiment, the server may also function as the user device, eliminating the need for a separate computing device for input and selection. This may be achieved by integrating a user interface directly into the server, facilitating users to access and interact with the customization platform through an embedded touchscreen, kiosk, or dedicated terminal. In such a configuration, the server may facilitate user input, process selections, generate the finalized design, and manage printing. Such approach may be beneficial for onsite implementations, such as live events or designated printing stations, where users can directly engage with the server to create and receive their personalized pocket cards without requiring additional devices.

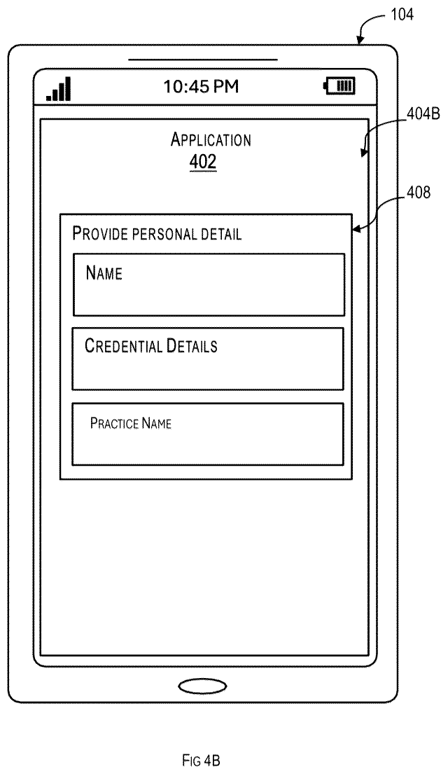

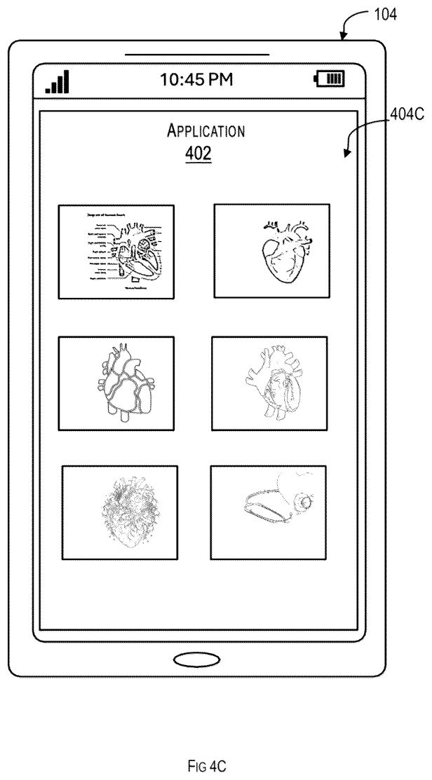

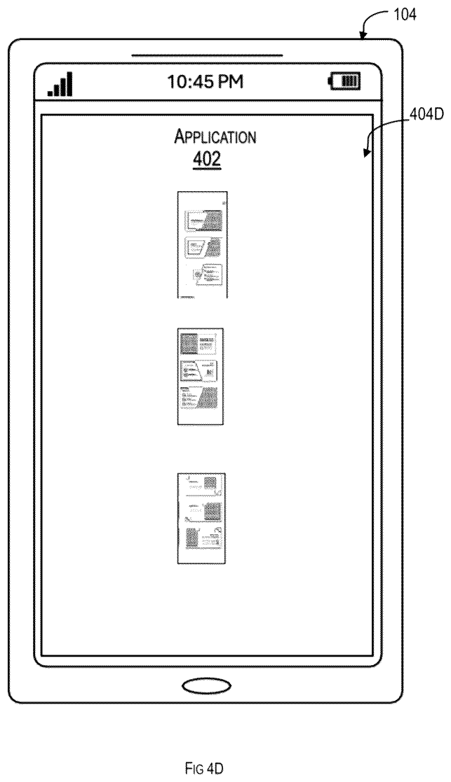

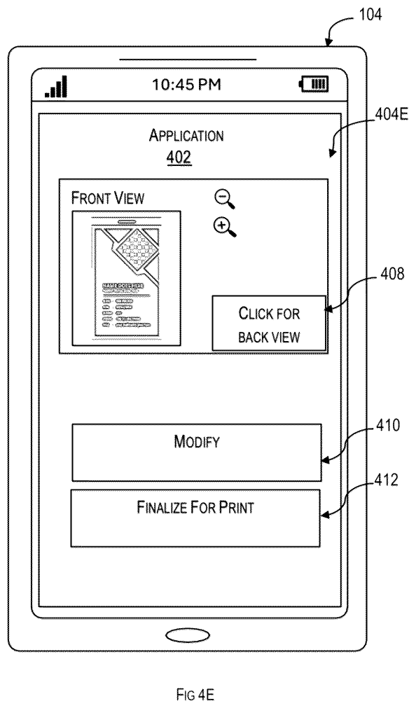

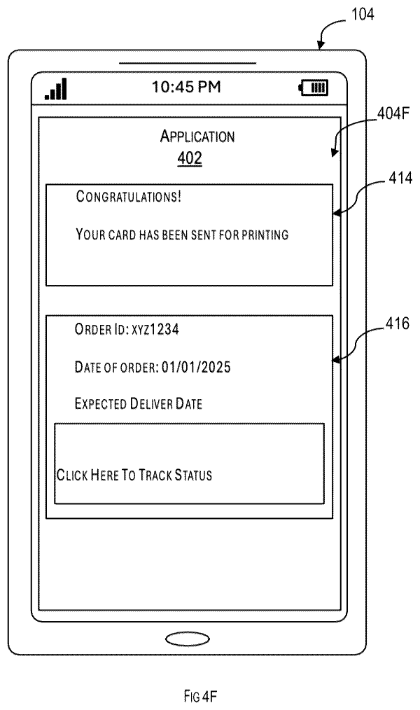

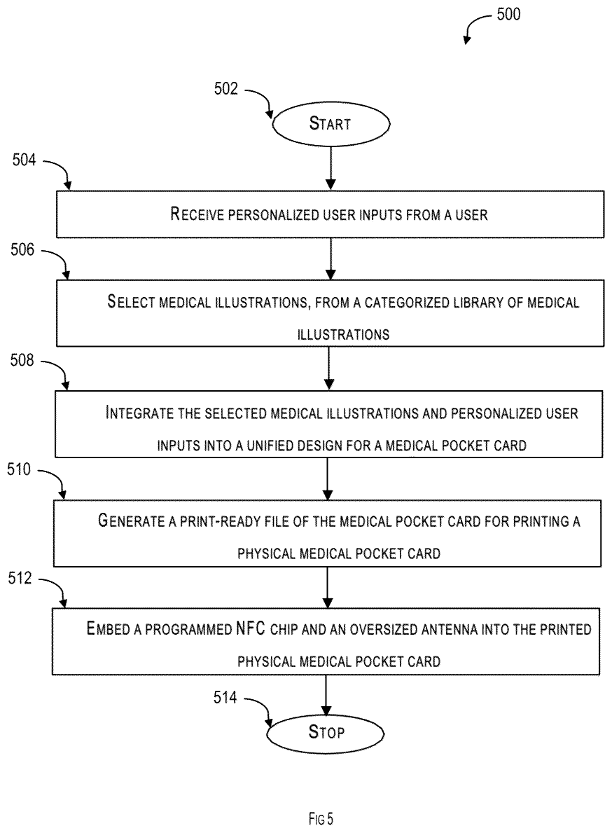

FIG. 4A-4F illustrate various exemplary user interfaces (404A, 404B, 404C 404D, 404E, and 404F) of an application 402 for creating personalized NFC enabled medical pocket card 114, in accordance with an embodiment of the present disclosure. For the sake of brevity, FIG. 4A-4F have been explained together.

In an embodiment, as shown in FIG. 4A, the user interface 404A of the user device 104 may facilitate the user 102 for creating a user account within the application 402. The user 102 may be prompted to enter essential details to login such as a unique user ID, password, and email address, as shown by 406. The user interface 404A may include form fields for these inputs, along with visual indicators, such as tooltips or placeholders, to guide the user through the process. Security features, such as password strength indicators and CAPTCHA verification, may be implemented to ensure account safety. Additionally, the user 102 may have options to reset forgotten passwords, agree to terms and conditions, or subscribe to notifications and updates about the application.

In an embodiment, as shown in FIG. 4B, the user interface 404B of the user device 104 may facilitate the user 102 to provide personalized details, including their name, credentials, practice name, and other professional identifiers, as shown by 408. The user interface 404B may include structured fields with validation checks to ensure accuracy and completeness, such as verifying that all mandatory fields are filled. The user 102 may also have the option to upload additional branding assets, such as a logo or custom background, to further personalize the medical pocket card 114. Error messages or prompts may appear if inputs are incomplete or do not meet predefined requirements, ensuring that the data provided is suitable for card customization.

In an embodiment, as shown in FIG. 4C, the user interface 404C of the user device 104 may provide access to the categorized library of medical illustrations. The library may be organized by topics such as anatomy, medical conditions, and procedures, with filtering and search options for ease of navigation. The user interface 404C may display visual thumbnails of available illustrations, along with metadata, such as titles or descriptions, to help the user 102 make informed selections. The user may preview individual illustrations before selecting them, ensuring they align with the intended purpose of the medical pocket card 114.

In an embodiment, as shown in FIG. 4D, the user interface 404D of the user device 104 may enable the user 102 to choose from multiple layout and artwork options for their medical pocket card 114. The interface may display a gallery of predefined layouts and themes, each showcasing a unique combination of text placement, image positioning, and overall design aesthetics. The user 102 may select a layout that aligns with their preferences and needs, such as a minimalistic design for professional use or a more creative theme for patient engagement. The user interface 404D may also allow the user 102 to customize elements, such as font styles, color schemes, or artwork placement, within the selected layout.

In an embodiment, as shown in FIG. 4E, the user interface 404E of the user device 104 may provide a preview of the card design. The user 102 may view a detailed preview of the front side of the pocket card 114, with an option 408 to toggle and preview the backside as well. Further, the user interface 404E may include tools to zoom in, inspect specific elements, and identify any areas requiring modification. The user 102 may also be provided an option 410 to modify individual components, such as text alignment, image placement, or color balance, directly from the preview interface. Further, the user 102 may have the option to go back and modify earlier inputs to ensure they meet their preferences. Once satisfied with the design, the user 102 may select an option 412 to finalize the card for printing. The interface may prompt a confirmation message to ensure that no further changes are required before proceeding.

In an embodiment, as shown in FIG. 4F, the user interface 404F of the user device 104 may provide a confirmation that the card design has been sent for printing. Further, the user interface 404F may display a congratulatory message, such as “Congratulations! Your card has been sent for printing,”, as shown by 414, along with details of the selected design. The user may also be provided with an order summary, as shown by 416, including the expected timeline for card production and delivery. Furthermore, the user interface 404F may guide the next steps, such as instructions for NFC embedding, once the printed card 114 is received. It will be understood by those skilled in the art that the sequence of user interfaces (404A, 404B, 404C, 404D, 404E, and 404F) is not fixed and may be varied based on the specific implementation or design requirements of the system.

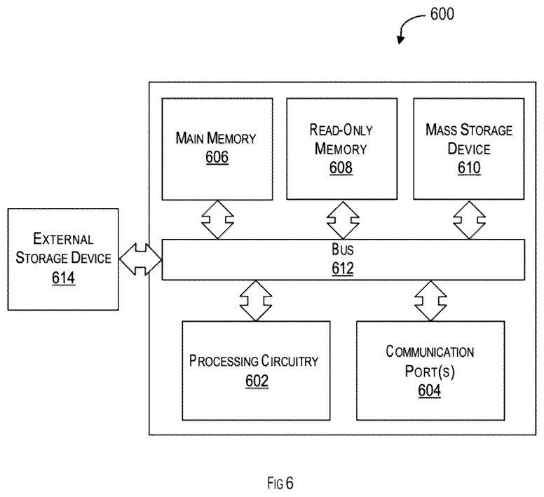

FIG. 5 is a flowchart 500 illustrating a method for creating personalized NFC enabled medical pocket card, in accordance with an embodiment of the present disclosure. For a person skilled in the art, steps in the method 500 may be performed in various orders depending on the specific implementation or design requirements. The method starts at step 502.

At step 504, the method may include receiving personalized user inputs from a user. The user inputs may include a name, a credential, and/or a practice name. Further, the user input may adhere to predefined formatting requirements, such as proper capitalization, character limits, and text alignment. Further, the method may include facilitating multilingual options, enabling the user to input text in their preferred language.

At step 506, the method includes selecting medical illustrations from a categorized library of medical illustrations. The categorized library may dynamically be presented based on the received user inputs. Further, the categorized library may be organized by topics including anatomy, medical conditions, and/or medical procedures. Further, the categorized library may be enriched with metadata to enable keyword-based searching and filtering for efficient navigation. In an embodiment, once selected, the medical illustrations may dynamically be adjusted to fit within predefined dimensions and layout constraints, ensuring compatibility with the card's design.

At step 508, the method may include integrating the selected medical illustrations and personalized user inputs into a unified design for a medical pocket card. The integration may be performed in a manner that ensures proper alignment and formatting, resulting in a professional and visually appealing layout.

At step 510, the method may include generating a print-ready file of the medical pocket card to print a physical medical pocket card. The print-ready file may be optimized for high-resolution output by applying color calibration, bleed margin adjustments, and other print settings to ensure compatibility with printing environments. Further, the print-ready file may be optimized for high-resolution output by applying color calibration, bleed margin adjustments, and other print settings. Further, the print-ready file may be generated in multiple formats to ensure compatibility with different production environments, such as thermal or ink-based printing systems.

At step 512, the method may include embedding a programmed NFC chip and an oversized antenna into the printed physical medical pocket card. The NFC chip may be pre-programmed with unique identifiers. The identifiers may include URL(s) and/or resource identifier that provides access to online educational materials, patient resources, or multimedia content. Further, the identifiers may provide access to online educational materials, patient resources, or multimedia content. The method ends at 514.

FIG. 6 illustrates an exemplary computer system 600 in which or with which embodiments of the present disclosure may be utilized. As shown in FIG. 6, a computer system 600 includes an external storage device 614, a bus 612, a main memory 606, a read-only memory 608, a mass storage device 610, a communication port 604, and a processor 602.

Those skilled in the art will appreciate that computer system 600 may include more than one processor 602 and communication ports 604. Examples of processor 602 include, but are not limited to, an Intel® Itanium® or Itanium 2 processor(s), or AMD® Opteron® or Athlon MP® processor(s), Motorola® lines of processors, FortiSOC™ system on chip processors or other future processors. The processor 602 may include various modules associated with embodiments of the present disclosure.

The communication port 604 can be any of an RS-232 port for use with a modem-based dialup connection, a 10/100 Ethernet port, a Gigabit or 10 Gigabit port using copper or fiber, a serial port, a parallel port, or other existing or future ports. The communication port 604 may be chosen depending on a network, such as a Local Area Network (LAN), Wide Area Network (WAN), or any network to which the computer system connects.

The memory 606 can be Random Access Memory (RAM), or any other dynamic storage device commonly known in the art. Read-Only Memory 808 can be any static storage device(s) e.g., but not limited to, a Programmable Read-Only Memory (PROM) chips for storing static information e.g., start-up or BIOS instructions for processor 602.

The mass storage 610 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7200 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

The bus 612 communicatively couples processor(s) 602 with the other memory, storage, and communication blocks. The bus 612 can be, e.g., a Peripheral Component Interconnect (PCI)/PCI Extended (PCI-X) bus, Small Computer System Interface (SCSI), USB, or the like, for connecting expansion card, drives, and other subsystems as well as other buses, such a front side bus (FSB), which connects processor 602 to a software system.

Optionally, operator and administrative interfaces, e.g., a display, keyboard, and a cursor control device, may also be coupled to bus 612 to support direct operator interaction with the computer system. Other operator and administrative interfaces can be provided through network connections connected through communication port 604. An external storage device 614 can be any kind of external hard-drives, floppy drives, IOMEGA® Zip Drives, Compact Disc Read-Only Memory (CD-ROM), Compact Disc-Re-Writable (CD-RW), Digital Video Disk Read Only Memory (DVD-ROM). The components described above are meant only to exemplify various possibilities. In no way should the aforementioned exemplary computer system limit the scope of the present disclosure.

One or more embodiments of the present disclosure relate to a system and method (together termed as mechanism) for creating personalized NFC-enabled medical pocket card. The objective of the mechanism is to address the challenges faced by healthcare professionals in bridging physical and digital resources, including the lack of personalized, interactive tools for patient engagement, inefficiencies in on-demand customization, and limited accessibility to multilingual and multi-media resources. By integrating advanced customization features, including dynamic content integration and real-time design previews, the mechanism aims to provide a seamless and efficient solution for creating interactive and professional medical pocket card.

Unlike traditional QR code-based systems, the mechanism described herein provides a more seamless, user-friendly, and secure approach for patient engagement and resource access. While QR codes require precise scanning and alignment, which can be difficult for individuals with visual, motor, or cognitive impairments, the NFC-enabled card offer a simple and intuitive experience. Users only need to bring their NFC-enabled device close to the card, without the need for visual alignment or manual interaction. This eliminates the risk of errors associated with QR code scanning, such as improper positioning or damage to the code, and enhances accessibility for all users, regardless of their physical or cognitive limitations. Furthermore, NFC technology provides a higher level of security compared to QR codes, reducing the risk of malicious tampering or redirection, which can be a concern with QR-based systems.

Further, the mechanism employs innovative features, such as dynamically adjustable medical illustrations and multilingual text integration, to ensure each card meets the unique requirements of healthcare professionals across various disciplines. The mechanism leverages preprogrammed NFC chips embedded into the card, allowing them to function as a gateway to online resources such as patient education materials, multimedia content, and professional tools. By supporting dynamic linking capabilities and accommodating diverse device compatibility with oversized NFC antennas, the mechanism ensures reliable and efficient NFC interactions across a wide range of smartphones.

The mechanism also excels in operational efficiency by offering on-demand production capabilities, including live-event printing and secure online ordering. Automated workflows streamline the card creation process, minimizing the time and effort required for manual adjust-ments. The card designs are optimized for high-resolution printing, ensuring a polished and professional output. Additionally, the integration of advanced quality checks ensures that every card meets the desired standards for formatting, alignment, and functionality. These capabilities enable healthcare professionals to focus more on patient care while receiving customized tools that meet their branding and educational needs.

On the patient engagement front, the mechanism enhances accessibility and interac-tion by integrating NFC technology, which enables seamless access to educational resources in multiple languages. The mechanism ensures inclusivity by supporting multilingual text inputs and facilitating cultural adaptability. By linking physical card to dynamic online content, the mechanism empowers healthcare professionals to deliver up-to-date and personalized patient education, fostering trust and understanding between patients and providers. The high degree of customization ensures that card cater to specific healthcare practices, reinforcing professional branding while improving communication and resource accessibility.

Overall, the mechanism provides an innovative and transformative solution for healthcare professionals seeking personalized and interactive tools to enhance patient engagement and professional branding. By combining technological advancements with practical healthcare needs, the mechanism offers a scalable and impactful approach to bridging physical and digital resources, addressing existing challenges in patient education, communication, and resource distribution.

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, utilized, or combined with other elements, components, or steps that are not expressly referenced. Where the specification claims refer to at least one of something selected from the group consisting of A, B, C . . . . And N, the text should be interpreted as requiring only one element from the group, not A plus N, or B plus N, etc.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions, or examples, which are included to enable a person having ordinary skill in the art to make and use the disclosure when combined with information and knowledge available to the person having ordinary skill in the art.

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions, or examples, which are included to enable a person having ordinary skill in the art to make and use the disclosure when combined with information and knowledge available to the person having ordinary skill in the art.

What is claimed is

1. A system for creating personalized Near Field Communication (NFC) enabled medical pocket card, the system comprising:
a receiving module configured to accept personalized user inputs including at least a name, a professional credential, and a practice identifier;
a content selection module configured to present and allow selection of medical illustrations from a categorized library organized by at least one of anatomy, medical conditions, and medical procedures, wherein the library is dynamically presented based on the received inputs;
a content integration module configured to combine the personalized user inputs and the selected medical illustrations into a unified digital design formatted to predefined card dimensions;
a print management module configured to generate a print-ready file from the unified design for high-resolution output; and
an embedding module configured to integrate a pre-programmed NFC chip and an over-sized NFC antenna into the print-ready physical card, the NFC chip being programmed with a unique URL or resource identifier that provides access to online educational materials, patient resources, or multimedia content.

2. The system of claim 1, further comprising:
a multilingual support module configured to validate and incorporate user inputs in a selected language; and
an analytics module configured to:
track usage of the NFC-enabled personalized medical cards at an event level;
identify medical illustrations most frequently selected across multiple events;
monitor engagement and traffic generated through the cards, including referral activity linked to specific medical practices, wherein the analytics module is further configured to operate without storing or processing any personally identifiable information or protected health information; and
perform personalization and delivery of medical content in less than 30 seconds from point of user interaction.

3. The system of claim 1, wherein the content selection module further comprises filtering means based on metadata associated with the medical illustrations.

4. The system of claim 1, wherein the content integration module dynamically adjusts the size and placement of the selected illustrations to conform to the card's layout requirements.

5. The system of claim 1, further including an authentication module that verifies the identity of the user before accepting personalization inputs.

6. The system of claim 1, wherein the embedding module locks the pre-programmed NFC chip after programming to prevent unauthorized modification of the stored URL or resource identifier.

7. The system of claim 1, wherein the personalized user inputs are received via a user interface deployed on a web-enabled device and transmitted over a secure communication network.

8. The system of claim 1, wherein the categorized library of medical illustrations is stored in and accessed from a database that supports keyword-based searches and filtering.

9. A method for creating a personalized NFC-enabled medical pocket card, comprising the steps of:
receiving personalized user inputs including a name, a professional credential, and a practice identifier;
presenting a categorized library of medical illustrations and selecting one or more illustrations based on the received inputs;
integrating the selected illustrations with the personalized user inputs to produce a unified digital design for the medical pocket card;
generating a print-ready file of the digital design optimized for high-resolution, variable data printing; and
embedding a pre-programmed NFC chip with an oversized antenna into the printed card, the NFC chip being programmed with a unique URL or resource identifier that links to online educational materials, patient resources, or multimedia content.

10. The method of claim 9, further comprising the step of providing a multilingual option that validates and incorporates the user's language selection into the digital design.

11. The method of claim 9, wherein the step of presenting the categorized library includes filtering options based on metadata tags corresponding to medical specialties.

12. The method of claim 9, further comprising the step of authenticating the user before accepting the personalized user inputs.

13. The method of claim 9, wherein the step of generating the print-ready file includes applying color calibration, bleed margin adjustments, and resolution optimization parameters.

14. The method of claim 9, wherein the step of embedding the NFC chip includes programming the NFC chip with a unique identifier and subsequently locking the chip to preclude reprogramming.

15. The method of claim 9, further comprising the step of dynamically previewing the unified digital design in real time prior to printing.

16. A personalized NFC-enabled medical pocket card, comprising:
a printed card displaying personalized text and medical illustrations that have been dynamically integrated into a unified design;
an embedded NFC chip programmed with a unique URL or resource identifier for accessing online educational materials, patient resources, or multimedia content; and
an oversized NFC antenna configured to enhance signal reliability across a plurality of NFC-enabled devices.

17. The personalized medical pocket card of claim 16, wherein the personalized text is presented in a language selected by the user.

18. The personalized medical pocket card of claim 16, wherein the NFC chip is pre-programmed and subsequently locked to prevent further modification of its stored information.

19. The personalized medical pocket card of claim 16, wherein the printed card is produced on demand using a variable data printing process optimized for high-resolution output.

20. The personalized medical pocket card of claim 16, wherein the card design is dynamically adjusted to maintain appropriate formatting, alignment, and image scaling within predefined dimensions.
